(19) United States
(12) Patent Application Publication
(10) Pub. No.: US 2025/0262170 A1
(43) Pub. Date: Aug. 21, 2025

(54) COMBINATIONS OF BETA 2-ADRENERGIC RECEPTOR AGONISTS AND BETA 3-ADRENERGIC RECEPTOR AGONISTS, AND MEDICAL USES THEREOF

(71) Applicant: ATROGI AB, Solna (SE)

(72) Inventor: Tore Bengtsson, Vaxholm (SE)

(21) Appl. No.: 18/858,544

(22) PCT Filed: Apr. 21, 2023
(86) PCT No.: PCT/EP2023/060502

§ 371 Date: Oct. 21, 2024

(30) Foreign Application Priority Data
Apr. 22, 2022 (GB) 2205895.2

(51) Int. Cl. A61K 31/136; A61K 31/137; A61K 31/36; A61P 3/04

(52) U.S. Cl. CPC A61K 31/136; A61K 31/137; A61K 31/36; A61P 3/04

(57) ABSTRACT

There is herein provided a pharmaceutical formulation comprising one or more compounds that are β₂-adrenergic receptor agonist, or a pharmaceutically acceptable salt thereof, and β₃-adrenergic receptor agonist, or a pharmaceutically acceptable salt thereof, and optionally one or more pharmaceutically acceptable excipient, for use in the treatment or prophylaxis of obesity and other medical uses.

### FIELD OF THE INVENTION

The present invention relates to methods for the treatment or prophylaxis of obesity and the reduction of body fat. In particular, the invention relates to methods for the treatment or prophylaxis of obesity and therapeutic and non-therapeutic methods of reducing body fat, involving activating both the β₂- and the β₃-adrenergic receptors, and to compounds and compositions for use in such methods.

### BACKGROUND OF THE INVENTION

The listing or discussion of an apparently prior-published document in this specification should not necessarily be taken as an acknowledgement that the document is part of the state of the art or is common general knowledge.

Energy dissipating mechanisms can exert a significant metabolic benefit by protecting against complications that often result from a chronic imbalance between energy intake and energy demands. The stimulation of brown adipose tissue (BAT) thermogenesis in humans has emerged as an attractive target to improve metabolic health.

The β-adrenergic receptors (β-ARs) are also divided into the subtypes, β₁, β₂, and β₃, of which β₂-AR is the major isoform in skeletal muscle cells. ARs are G protein coupled receptors (GPCRs) which signal through classical secondary messengers, such as cyclic adenosine monophosphate (cAMP).

Based on murine studies, it was thought that pharmacological stimulation targeting the β₃-adrenergic receptor mediates BAT thermogenesis. However, agonists of the β₃ receptor have not performed well in human clinical trials.

It was recently found that a β₃-adrenergic receptor agonist, mirabegron, elicits increased BAT thermogenesis only when ingested at the maximal allowable dosage, resulting from off-target binding to β₁-adrenergic receptor and β₂-adrenergic receptor, in turn leading to increased cardiovascular responses and white adipose tissue lipolysis, respectively (see Blondin et al., Cell Metabolism, 32, 287-300 (2020)). In the same study it was shown that in human BAT it is the β₂-adrenergic receptor which mediates thermogenesis.

It has also been found that, in humans, lipids may be burned in brite (beige) adipose tissue. White adipocytes may also be converted to brite adipocytes, which may be converted to brown fat (browning) (see Evans et al., Mol. Nutr. Food Res., 60, 18-42 (2016)).

Furthermore, it was recently found that treatment with a β₂-adrenoceptor agonist stimulates glucose uptake in skeletal muscle and improves glucose homeostasis, insulin resistance and hepatic steatosis in mice with diet-induced obesity (see Kalinovich et al., Diabetologia. 63(8), 1603-1615 (2020)).

Thus, pharmacological activation of the β₂-adrenergic receptor has been suggested to provide a means for inducing BAT thermogenesis, which in turn would result in a reduction in adipose tissue due to lipolysis.

On the other hand, it is known that β₃-adrenergic receptors are involved in lipolysis and thermogenesis, and the use of β₃-adrenergic receptor agonists for the treatment of disorders such as obesity and type 2 diabetes has been extensively studied (see, for example, Zhu et al. Biorg. Med. Chem. Lett. (2016) 55-59).

WO 2004/110375 discloses a combination therapy based on the use of an anti-obesity agent and an anti-diabetes agent for the treatment of diabetes, wherein β₃-adrenergic receptor agonists are classified amongst the agents as anti-obesity agents for use in the co-therapy.

Thus, various publications have suggested the use of either β₂- or β₃-adrenergic receptor agonists separately in the treatment of conditions characterized by excess body weight, such as obesity. However, there has been no teaching or suggestion of such a treatment based on the effect of using such agents in combination.

There remains, therefore, a need for new treatments capable of improving metabolic health, such as through the treatment or prophylaxis of obesity and the reduction of body fat.

### DESCRIPTION OF THE INVENTION

We have now surprisingly found that a combination of activation of the β₂-adrenergic receptor and the β₃-adrenergic receptor represents a promising strategy for the treatment and prophylaxis of conditions characterized by excess body weight, such as obesity and metabolic syndrome, and dyslipidaemia, and for the reduction of body fat, both for therapeutic and non-therapeutic purposes (e.g. cosmetic methods).

Furthermore, it is possible to utilise certain compounds acting as β₂-adrenergic receptor agonists, or in some cases as agonists of the β₂ and β₃ adrenergic receptors, which are able to activate the β₂-adrenergic receptor without (or with only a minimal effect in) inducing cAMP production.

### Pharmaceutical Formulations for Use

Accordingly, in a first aspect of the invention, there is provided a pharmaceutical formulation comprising one or more compounds that are:
(a) a β₂-adrenergic receptor agonist, or a pharmaceutically acceptable salt thereof; and
(b) a β₃-adrenergic receptor agonist, or a pharmaceutically acceptable salt thereof,
and optionally one or more pharmaceutically acceptable excipient, for use in:
(i) the treatment or prophylaxis of obesity;
(ii) a therapeutic method of lowering body fat composition and/or reducing body weight;
(iii) the treatment or prophylaxis of metabolic syndrome; or
(iv) the treatment or prophylaxis of dyslipidaemia,
which formulations may be referred to hereinafter as the “formulations of the invention”, or the like, and which treatments (and associated prophylaxis) may be referred to herein after as the “treatments of in the invention”, or the like.

Unless indicated otherwise, all technical and scientific terms used herein have the same meaning as commonly understood by one of ordinary skill in the art to which this invention pertains.

Preferences and options for a given aspect, embodiment, feature or parameter of the invention should, unless the context indicates otherwise, be regarded as having been disclosed in combination with any and all preferences and options for all other aspects, features and parameters of the invention.

Wherever the word “about” is employed herein (for example, in the context of doses of active ingredients) it will be appreciated that such variables are approximate and as such may vary by ±10%, for example ±5% and preferably ±2% (e.g. ±1%) from the numbers specified herein.

Wherever the word “optionally” is employed in relation to features described herein it will take its normal meaning, namely that the relevant feature may or may not be present.

Accordingly, in an alternative first aspect of the invention, there is provided a method for:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia,
comprising administering to a patient in need thereof a therapeutically effective amount of a pharmaceutical formulation comprising one or more compounds that are:
(a) a β₂-adrenergic receptor agonist, or a pharmaceutically acceptable salt thereof; and
(b) a β₃-adrenergic receptor agonist, or a pharmaceutically acceptable salt thereof,
and optionally one or more pharmaceutically acceptable excipient.

The skilled person will understand that compounds referred to herein, such as compounds referred to as agonists, may be provided in the form of a pharmaceutically acceptable salt.

Pharmaceutically acceptable salts include acid addition salts and base addition salts, each of which may be in the form of salts in varying ratios of compound to counter ion (e.g. including hemi salts). Such salts may be formed by conventional means, for example by reaction of a free acid or a free base form of a compound comprised in the formulations of the invention with one or more equivalents of an appropriate acid or base, optionally in a solvent, or in a medium in which the salt is insoluble, followed by removal of said solvent, or said medium, using standard techniques (e.g. by rotary evaporation under reduced pressure, by freeze-drying or by filtration). Salts may also be prepared by exchanging a counter-ion of a compound comprised in the formulations of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, a-hydroxybutyrate, lactate, tartrate, hemi-tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, 1-naphthoate, 2-naphthoate, 1-hydroxy-2-naphtoate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1,2-ethanedisulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, compounds suitable for use in the formulations, and other aspects, of the invention (e.g. β₂- and β₃-adrenergic receptor agonists, such as those described herein) may exist as solids, and thus the scope of the invention includes all amorphous, crystalline and part crystalline forms thereof, and may also exist as oils.

Where such compounds exist in crystalline and part crystalline forms, such forms may include solvates, which are included in the scope of the invention. The compounds may also exist in solution.

Suitable pharmaceutical formulations may be commercially available or otherwise are described in the literature, such as, Remington, The Science and Practice of Pharmacy, 19th ed., Mack Printing Company, Easton, Pennsylvania (1995), and Martindale—The Complete Drug Reference (35^{th} Edition), and the documents referred to therein, the relevant disclosures in all of which documents are hereby incorporated by reference in their entirety. Otherwise, the preparation of suitable formulations, and in particular combined preparations including both a β₂- and a β₃-adrenergic receptor agonist, or pharmaceutically acceptable salts thereof, may be achieved by the skilled person using routine techniques.

References to pharmaceutically acceptable excipient(s) may be understood to include pharmaceutically acceptable, diluents, carriers and/or adjuvants, as known to those skilled in the art.

The skilled person will understand that references to an agonist will refer to compounds suitable for acting as such when administrated to a subject to be treated (i.e. a patient, e.g. a human, in need thereof). Suitable compounds may include compounds which provide the required effect and compounds which are converted to compounds providing the required effect after administration (i.e. in vivo), which compounds may be referred to as pro-drugs. Particular compounds that may be mentioned are compounds which elicit the required effect.

For the avoidance of doubt, the term “agonist” may be understood to indicate an agent (i.e. a compound) that induces activation of the relevant receptor to produce a biological response (e.g. in a subject, such as a human), such as by binding to the relevant receptor. As such, the term may also refer to partial agonists (which will be understood to refer to compounds that activate a given receptor, but have only partial efficacy at the receptor relative to a full agonist).

Agonists (and partial agonists) may display, for example, half maximal effective concentration (EC₅₀) values of less than about 1 mM, such as less than about 100 μM, or less than about 10 μM, such as less than about 1 μM (e.g. less than about 100, about 10 or about 1 nM).

Unless otherwise stated or clear from the context, references herein to agonists will also include pharmaceutically-acceptable (e.g. “protected”) derivatives of compounds which may not possess the relevant activity per se, but may be administered (e.g. parenterally or orally) to a patient and thereafter be metabolised in the body to form compounds possessing the required activity, which compounds may be referred to as prodrugs. Suitable prodrugs of compounds as described herein will be known to those skilled in the art, such as suitable esters (e.g. methyl or ethyl esters, and the like).

For the avoidance of doubt, unless otherwise stated or clear from the context, references to compounds that are agonists, and pharmaceutically acceptable salts thereof, will include compounds that are prodrugs of such agonists, and pharmaceutically acceptable salts thereof.

Suitable β₂- and β₃-adrenergic receptor agonists will be known to those skilled in the art. Further, for a given β₂- or β₃-adrenergic receptor agonist, the skilled person will be able to determine whether the compound will also act as a suitable agonist of the other receptor (i.e. whether the compound will act as a suitable β₂- and β₃-adrenergic receptor agonist).

In certain embodiments, suitable β₂- or β₃-adrenergic receptor agonists will include those that are selective, which term will be known to those skilled in the art (i.e. compounds that are agonists of the relevant receptor(s) but which do not cause significant activation of other β-adrenergic receptors). For the avoidance of doubt, in the case of compounds acting both as a suitable β₂- and β₃-adrenergic receptor agonist, the term selective will indicate that such compounds are agonists of both receptors but do not cause significant activation of other β-adrenergic receptors.

Suitable β₂-adrenergic receptor agonists can be identified using techniques known to those skilled in the art, including those as described in the examples provided herein.

Suitable β₂-adrenergic receptor agonists that may be employed in the various aspects of the invention include, but are not limited to, those described in: WO 2004/071388, EP 0 272 976, FR 2647310, DE 2 157 040, DE 2212600, DE 2015573, ZA 6705591, DE 2128258, WO 91/09596, GB 1 199 630, DE 4209989, BE 611502, NL 7804582, EP 0 043 807, WO 2008/022038, DE 2413102, U.S. Pat. No. 2,308,232, BE 823841, BE 660244, WO 2000/075114, WO 2005/102350, WO 2005/110990, JP 56055355, AT 285583, U.S. Pat. Nos. 4,223,137, 3,056,836, FR 1324914, DE 638650, DD 45721, U.S. Pat. No. 3,801,631, DE 2259282, DE 2300614, EP 0 290 122, US 2004/0266867, US 2010/0022658, US 2010/0022659, DE 2157040, GB 2133986, WO 2006/122788, Woo et al., Molecular Pharmacology, (2009), 75(1) 158-165, Baur et al., J. Med. Chem., (2010), 53(9), 3675-3684, Kaiser et al., J. Med. Chem. (1974), 17(1) 49-57, Baker et al., J. Pharmacology and Experimental Therapeutics, (2006), 319(1), 439-446, Engelhardt et al., Arzneimittel-Forschung, (1972), 22(5), 869-76, WO 2019/241744, WO 2019/241736, WO 2020/198466, WO 2021/003161, WO 2021/081292, WO 2021/127210 and WO 2021/247934, the relevant disclosures of each of which (e.g. the examples compounds described therein, and pharmaceutically acceptable salts thereof, and associated methods of preparation) are hereby incorporated by reference in their entirety.

For the avoidance of doubt, references to patent publications will typically refer to the initial publication of the full patent specification with the relevant publication number (which may be indicated by the suffix “A1”).

Further suitable β₂-adrenergic receptor agonists that may be employed in the various aspects of the invention (which compounds may be identified as also being suitable β₂-adrenergic receptor agonists) include those described in the following publications, the contents of which are hereby incorporated herein in their entirety (in particular, the biological examples, the generic compound definitions, including all embodiments thereof and associated definitions, and the example compounds provided therein, including pharmaceutically acceptable salts thereof, and associated methods of preparation):
WO 2017/153737
WO 2019/053429
WO 2019/053426
WO 2019/053425
WO 2019/053427
WO 2020/188299
WO 2020/188301
WO 2022/063895
WO 2022/063889
WO 2023/046885
WO 2023/046882

Particular β₂-adrenergic receptor agonists as described in the above-mentioned publications that may be mentioned include, but are not limited to, the following:

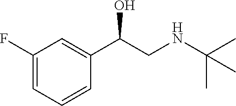

and pharmaceutically acceptable salts thereof.

In certain embodiments the invention, the β₂-adrenergic receptor agonist is selected from the group consisting of formoterol, arformoterol, salmeterol, (R)-salmeterol, vilanterol, zilpaterol, clenbuterol, (R)-clenbuterol, bitolterol, salbutamol, levosalbutamol, terbutaline, metaproterenol, pirbuterol, bambuterol, fenoterol, methoxyfenoterol, isoprenaline, procaterol, ritodrine, indacaterol, olodaterol, colterol, hexaprenaline, carmoterol, isoxsuprine, isoetarine, zinterol, bamethane, (R)-bamethane, clencyclohexerol, tulobuterol, BRL-47672, trantinterol, clenproperol, clenpenterol, brombuterol, ractopamine and abediterol, and pharmaceutically acceptable salts thereof.

In further certain embodiments, the β₂-adrenergic receptor agonist is selected from the group consisting of formoterol, arformoterol, salmeterol, (R)-salmeterol, vilanterol, zilpaterol, clenbuterol, (R)-clenbuterol, bitolterol, salbutamol, levosalbutamol, terbutaline, metaproterenol, pirbuterol, bambuterol, fenoterol, methoxyfenoterol, isoprenaline, procaterol, ritodrine, indacaterol, olodaterol, colterol, hexaprenaline, carmoterol, isoxsuprine, isoetarine, zinterol, bamethane, (R)-bamethane, clencyclohexerol, tulobuterol, BRL-47672 and trantinterol, and pharmaceutically acceptable salts thereof.

In particular embodiments, the β₂-adrenergic receptor agonist is selected from the group consisting of formoterol, arformoterol, salmeterol, (R)-salmeterol, vilanterol, zilpaterol, clenbuterol, (R)-clenbuterol, indacaterol, olodaterol, carmoterol, bamethane, (R)-bamethane, clencyclohexerol, tulobuterol, trantinerol and abediterol, and pharmaceutically acceptable salts thereof.

In further particular embodiments, the β₂-adrenergic receptor agonist is selected from the group consisting of formoterol, arformoterol, salmeterol, (R)-salmeterol, vilanterol, zilpaterol, clenbuterol, (R)-clenbuterol, indacaterol, olodaterol, carmoterol, bamethane, (R)-bamethane, clencyclohexerol, tulobuterol and trantinerol, and pharmaceutically acceptable salts thereof.

In more particular embodiments, the β₂-adrenergic receptor agonist is selected from the group consisting of formoterol, arformoterol, salmeterol, (R)-salmeterol, clenbuterol, (R)-clenbuterol, bamethane, (R)-bamethane, trantinerol and abediterol, and pharmaceutically acceptable salts thereof.

In yet more particular embodiments, the β₂-adrenergic receptor agonist is selected from the group consisting of formoterol, arformoterol, salmeterol, (R)-salmeterol, clenbuterol, (R)-clenbuterol, bamethane, (R)-bamethane and trantinerol, and pharmaceutically acceptable salts thereof.

For the avoidance of doubt, the structures of bamethane (CAS: 3703-79-5) and (R)-bamethane (CAS: 912804-58-1) are shown below.

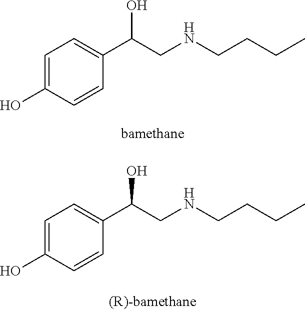

For the avoidance of doubt, in the case of a discrepancy between the name of the compound and the structure drawn in this specification, the structure should prevail.

For the avoidance of doubt, in the case of a discrepancy between the name of the compound and the structure drawn in this specification, the structure should prevail.

In further embodiments, the β₂-adrenergic receptor agonist is selected from the group consisting of formeterol, arformeterol, salmeterol, clenbuterol, tulobuterol, bambuterol vilanterol, indacaterol, olodaterol, carmoterol and abediterol, and pharmaceutically acceptable salts thereof.

In yet further embodiments, the β₂-adrenergic receptor agonist is selected from the group consisting of salbutamol, ritodrine, colterol, hexaprenaline and isoxsuprine, and pharmaceutically acceptable salts thereof.

In particular embodiments, the β₂-adrenergic receptor agonist is clenbuterol, or a pharmaceutically acceptable salt thereof.

For the avoidance of doubt, the compound clenbuterol may be understood to have the following structure:

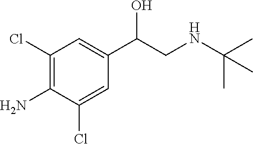 [SMILES: CC(C)(C)NCC(O)c1cc(Cl)c(N)c(Cl)c1]

Suitable β₃-adrenergic receptor agonists can be identified using techniques known to those skilled in the art (see, for example, Ujiantari et al., Molecular Informatics (2021) DOI 10.1002/minf.202100223, the contents of which, including the compounds described therein and pharmaceutically acceptable salts thereof, are incorporated herein in their entirety), including those as described in the examples provided herein.

Suitable β₃-adrenergic receptor agonists that may be employed in the various aspects of the invention include those described in: EP 0 023 385, WO 99/20607, EP 0 303 546, EP 0 436 435, WO 99/65877, WO 2009/124166, WO 2009/124167, WO 2011/025960, WO 98/32753, EP 1 095 932, U.S. Pat. No. 5,061,727, DE 2700193, DE 2819458, EP 0 611 003, WO 96/04234, WO 98/22480, U.S. Pat. No. 4,927,836, WO 93/15041, U.S. Pat. No. 5,705,515, WO 01/74782, WO 02/232897, EP 0 659 737, EP 0 357 956, WO 97/25311, US 2003/0018061, U.S. Pat. No. 4,743,604, Harper et al., Biorg. Med. Chem. Lett. (2017), 27, 1094, Edmondson et al., J. Med. Chem. (2016), 59, 609, Zhu et al., Biorg. Med. Chem. Lett. (2016), 26, 55, Mathwink et al., J. Med. Chem. (2000), 43, 3832, Fisher et al., Biorg. Med. Chem. Lett. (1996) 6, 2253, Parmee et al., Biorg. Med. Chem. Lett. (1998), 8, 1107, Sennit et al., J. Pharmacol. Exp. Ther. (1998), 285, 1084-1095 and Gavai et al., Biorg. Med. Chem. Kett. (2001) 9, 3041 (the relevant disclosures of which, including the compounds described therein and pharmaceutically acceptable salts thereof, are hereby incorporated by reference in their entirety).

In certain embodiments of the invention, the β₃-adrenergic receptor agonist is selected from the group consisting of BRL-37344, BRL-35135, mirabegron, amirabegron, SR59104A, SR59119A, solabegron, vibegron, CAS: 1269433-49-9, CAS: 1269433-05-7, MK-0634, ritobegron, BMS-187257, CL 316243, CGP 12177, L-755,507, L-742,791, L-750,355, L-749,372, SB-226552, SB-251023, ICI-D 7114, FR 149175, Ro40-2148, CAS: 769118-12-9, rafabegron, BMS-196085, trecadrine, SB-418790, CHEMBL32599, CHEMBL75604, CHEMBL22318, CHEMBL127656, CHEMBL22375, CHEMBL12769, CHEMBL331744 and CAS: 99151-51-6, and pharmaceutically acceptable salts thereof.

In particular embodiments, the β₃-adrenergic receptor agonist is selected from the group consisting of CL-316243, BRL-37344, BRL-35135, mirabegron, amirabegron, solabegron, vibegron, CAS: 1269433-49-9, CAS: 1269433-05-7 and ritobegron, and pharmaceutically acceptable salts thereof.

In further particular embodiments, the β₃-adrenergic receptor agonist is selected from the group consisting of CL-316243, mirabegron, vibegron, CAS: 1269433-49-9 and CAS: 1269433-05-7, and pharmaceutically acceptable salts thereof.

In a particular embodiment of the invention, the β₃-adrenergic receptor agonist is CL-316243, or a pharmaceutically acceptable salt thereof, such as the disodium salt (CAS number 138908-40-4; see the disclosure in Yoshida et al., Life Sciences, 54, 97 (1974), the contents of which are incorporated herein in their entirety).

For the avoidance of doubt, the compound CL-316243 may also be present in non-salt form or in the form of any pharmaceutically acceptable salt thereof. The compound CL-316243 (in non-salt form) is understood to have the following structure:

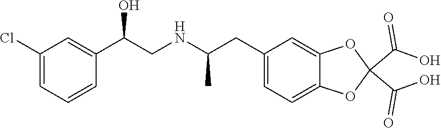 [SMILES: C[C@H](Cc1ccc2c(c1)OC(C(=O)O)(C(=O)O)O2)NC[C@H](O)c1cccc(Cl)c1]

As described herein, the β₂- and β₃-adrenergic receptor agonists employed in the formulations of the invention may typically be separate compounds, i.e. two distinct compounds, where each has activity (at least primarily or, in particular, specifically e.g. selective activity) at a single receptor subtype. However, in certain instances, the β₂- and β₃-adrenergic receptor agonists may also take the form of a single compound displaying activity (e.g. selective activity) as an agonist for both the β₂- and β₃-adrenergic receptor subtypes.

Thus, for the avoidance of doubt, the β₂-adrenergic receptor agonist and the β₃-adrenergic receptor agonist may be separate compounds or the same compound.

In certain embodiments, the formulations of the invention comprise a compound that is a β₂-adrenergic receptor agonist and another (i.e. a separate and chemically different) compound that is a β₃-adrenergic receptor agonist (including, in relation to both compounds, pharmaceutically acceptable salts thereof).

In particular embodiments, the β₂-adrenergic receptor agonist and the β₃-adrenergic receptor agonist are separate compounds, such as wherein:
the β₂-adrenergic receptor agonist is clenbuterol, or a pharmaceutically acceptable salt thereof; and/or (e.g. and)
the β₃-adrenergic receptor agonist is CL-316243, or a pharmaceutically acceptable salt thereof.

In further embodiments:
the β₂-adrenergic receptor agonist is

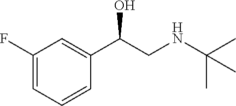

or a pharmaceutically acceptable salt thereof; and/or (e.g. and) the β₃-adrenergic receptor agonist is CL-316243, or a pharmaceutically acceptable salt thereof.

In certain embodiments, the formulations of the invention comprise a compound that is both a β₂-adrenergic receptor agonist and a β₃-adrenergic receptor agonist (i.e. a compound having at least both activities, which may be referred to as a dual active compound), including pharmaceutically acceptable salts thereof.

Thus, compounds referred to herein as a β₂-adrenergic receptor agonist may also be a β₃-adrenergic receptor agonist. Similarly, compounds referred to herein as a β₃-adrenergic receptor agonist may also be a β₂-adrenergic receptor agonist.

The skilled person will be able to identify a compound that is both a β₂-adrenergic receptor agonist and a β₃-adrenergic receptor agonist using techniques as known to those skilled in the art, such as those described herein.

For example, the skilled person may identify compounds which are both a β₂-adrenergic receptor agonist and a β₃-adrenergic receptor agonist by:
(i) identifying compound that is an agonist of the β₂- or β₃-adrenergic receptor (e.g. based on the prior disclosure of such activity, for example in the disclosures as referred to herein);
(ii) testing whether said compound is also an agonist of the other of the β₂- or β₃-adrenergic receptors (e.g. using techniques as described herein),
where compounds found to be an agonist of the β₂- and β₃-adrenergic receptor may be used in the methods of treatment and prophylaxis as described herein.

For the avoidance of doubt, compounds referred to herein (including those described in publications referred to herein) as β₂- or β₃-adrenergic receptor agonists may be identified (e.g. using techniques as described herein) as being compounds that act as an agonist of the β₂- and β₃-adrenergic receptors.

Particular compounds that are both a β₂-adrenergic receptor agonist and a β₃-adrenergic receptor agonist that may be mentioned include, but are not limited to, the following:

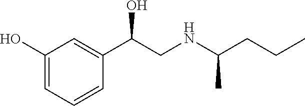 [SMILES: CCC[C@@H](C)NC[C@H](O)c1cccc(O)c1]

and pharmaceutically acceptable salts thereof.

For the avoidance of doubt, the international nonpropriety name (INN) or developmental drug code (e.g. BRL-37344) for a compound generally indicates the stereochemical configuration of the compound, or a particular mixture of stereoisomers (e.g. a racemate). Within the scope of the present invention, where relevant and unless context indicates otherwise (for example where both the racemate and a single stereoisomer are explicitly named), such names may also be considered to encompass separate stereoisomers that display the relevant biological activity, and which have not presently been assigned an alternative INN or developmental drug code.

In particular embodiments, the INN or developmental drug code should be understood to represent the compound to which the relevant name or code has been assigned only.

Where no INN or developmental drug code is available for a compound, the compound may be identified by its Chemical Abstracts Service Registry Number (CAS number).

As referred to herein, the indication “CAS: XXXXXX-XX-X” (wherein the number of figures in the first group may vary) is used to identify such compounds. Where relevant and unless context indicates otherwise, the CAS number for a compound may also be considered to encompass other stereoisomers, or mixtures thereof, that display the relevant biological activity, and which have not presently been assigned alternative CAS numbers (as described above for INNs and developmental drug codes).

In particular embodiments, the CAS number should be understood to represent the compound to which the relevant name or code has been assigned only.

The present invention also embraces pharmaceutical formulations comprising isotopically-labelled compounds, which are identical to the β₂- and β₃-receptor agonists recited herein but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the invention also encompasses pharmaceutical formulations comprising deuterated compounds, i.e. in which one or more hydrogen atoms are replaced by the hydrogen isotope deuterium.

As described herein, certain compounds acting as β₂-adrenergic receptor agonists, or in some cases as agonists of the β₂- and β₃-adrenergic receptors, are able to activate the β₂-adrenergic receptor without (or with only a minimal effect in) inducing cAMP production.

Thus, in particular embodiments, the methods and uses as described herein may be performed without inducing (or without inducing significant levels of) cAMP production.

The skilled person will be able to determine the level of cAMP production provided by compounds, such as those referred to herein, using techniques known to those skilled in the art, such as those described in the examples as provided herein.

Particular compounds acting as β₂-adrenergic receptor agonists, or in some cases as agonists of the β₂- and β₃-adrenergic receptors, which are able to activate the β₂-adrenergic receptor without (or with only a minimal effect in) inducing cAMP production include those described in the following publications, the contents of which are incorporated herein in their entirety (in particular, the biological examples, the generic compound definitions, including all embodiments thereof and associated definitions, and the example compounds provided therein, including pharmaceutically acceptable salts thereof, and associated methods of preparation):
WO 2017/153737
WO 2019/053429
WO 2019/053426
WO 2019/053425
WO 2019/053427
WO 2020/188299
WO 2020/188301
WO 2022/063895
WO 2022/063889
WO 2023/046885
WO 2023/046882

Particular compounds acting as agonists of both the β₂- and β₃-adrenergic receptors will include those described in the examples provided herein (in particular, Compound B and the compounds described in Biological Example 6).

### Kits-of-Parts

In a second aspect of the invention, there is provided a kit-of-parts comprising components:
(A) a pharmaceutical formulation comprising a β₂-adrenergic receptor agonist, or a pharmaceutically acceptable salt thereof, optionally in admixture with one or more pharmaceutically acceptable excipient, and
(B) a pharmaceutical formulation comprising a β₃-adrenergic receptor agonist, or a pharmaceutically acceptable salt thereof, optionally in admixture with one or more pharmaceutically acceptable excipient; or
(C) a pharmaceutical formulation comprising a compound that is a β₂-adrenergic receptor agonist, or a pharmaceutically acceptable salt thereof, and a β₃-adrenergic receptor agonist, or a pharmaceutically acceptable salt thereof, optionally in admixture with one or more pharmaceutically acceptable excipient,
which components (A) and (B) are each provided in a form that is suitable for administration in conjunction with the other, for use in:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia.

In an alternative second aspect of the invention, there is provided a kit-of-parts comprising:
(I) one of components (A) or (B) as defined hereinabove, and
(II) instructions to use that component in conjunction with the other of the two components; or
(III) component (C) as defined herein above, for use in:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia.

The kits-of-parts of the invention may be referred to hereinafter as the “kits-of-parts of the invention”.

For the avoidance of doubt, kits-of-parts of the second aspect of the invention may have any of the particular features described above for the first aspect of the invention, including all combinations thereof.

Thus, in relation to such kits-of-parts, it may be stated that the β₂-adrenergic receptor, or pharmaceutically acceptable salt thereof, agonist and the β₃-adrenergic receptor agonist, or pharmaceutically acceptable salt thereof, may be separate compounds or the same compound.

In certain embodiments, the kits-of-parts described herein may comprise more than one formulation including an appropriate quantity/dose of a β₂-adrenergic receptor agonist, or pharmaceutically acceptable salt and/or pro drug thereof, and/or more than one formulation including an appropriate quantity/dose of β₃-adrenergic receptor agonist, or pharmaceutically acceptable salt and/or pro drug thereof, in order to provide for repeat dosing. If more than one formulation (comprising either active compound) is present, such formulations may be the same, or may be different in terms of the dose of either compound, chemical composition(s) and/or physical form(s).

With respect to the kits-of-parts as described herein, by “administration in conjunction with” (and similarly “administered in conjunction with”) we include that respective formulations comprising a β₂-adrenergic receptor agonist, or pharmaceutically acceptable salt and/or pro drug thereof, and a β₃-adrenergic receptor agonist, or pharmaceutically acceptable salt and/or pro drug thereof, are administered, sequentially, separately or simultaneously, as part of a medical intervention directed towards treatment of the relevant condition.

Thus, in relation to the present invention, the term “administration in conjunction with” (and similarly “administered in conjunction with”) includes that the two active ingredients (i.e. a β₂-adrenergic receptor agonist, or pharmaceutically acceptable salt and/or pro drug thereof, and a β₃-adrenergic receptor agonist, or pharmaceutically acceptable salt and/or pro drug thereof) are administered (optionally repeatedly) either together, or sufficiently closely in time, to enable a beneficial effect for the patient, that is greater, over the course of the treatment of the relevant condition, than if either a formulation comprising a β₂-adrenergic receptor agonist, or pharmaceutically acceptable salt and/or pro drug thereof, or a formulation comprising a β₃-adrenergic receptor agonist, or pharmaceutically acceptable salt and/or pro drug thereof, are administered (optionally repeatedly) alone, in the absence of the other component, over the same course of treatment. Determination of whether a combination provides a greater beneficial effect in respect of, and over the course, of treatment of a particular condition will depend upon the condition to be treated or prevented, but may be achieved routinely by the skilled person.

Further, in the context of the present invention, the term “in conjunction with” includes that one or other of the two formulations may be administered (optionally repeatedly) prior to, after, and/or at the same time as, administration of the other component.

When used in this context, the terms “administered simultaneously” and “administered at the same time as” include that individual doses of a β₂-adrenergic receptor agonist, or pharmaceutically acceptable salt and/or pro drug thereof, and a β₃-adrenergic receptor agonist, or pharmaceutically acceptable salt and/or pro drug thereof, are administered within 48 hours (e.g. within 24 hours, 12 hours, 6 hours, 3 hours, 2 hours, 1 hour, 45 minutes, 30 minutes, 20 minutes or 10 minutes) of each other.

### Methods of Medical Treatment

In a third aspect of the invention there is provided a β₂-adrenergic receptor agonist, or a pharmaceutically acceptable salt thereof, for use in:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia,
wherein the use also comprises administration of a β₃-adrenergic receptor agonist, or a pharmaceutically acceptable salt thereof.

In an alternative third aspect of the invention there is provided a β₃-adrenergic receptor agonist, or a pharmaceutically acceptable salt thereof, for use in:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia,
wherein the use also comprises administration of a β₂-adrenergic receptor agonist, or a pharmaceutically acceptable salt thereof.

In a further alternative third aspect of the invention there is provided a method for:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia,
comprising administration of a therapeutically effective amount of a β₂-adrenergic receptor agonist, or a pharmaceutically acceptable salt thereof, and administration of a therapeutically effective amount of a β₃-adrenergic receptor agonist, or a pharmaceutically acceptable salt thereof, to a patient in need thereof.

For the avoidance of doubt, uses and methods of the third aspect of the invention may have any of the particular features described above for the first aspect of the invention, including all combinations thereof.

Thus, for the avoidance of doubt, references to a β₂- and β₃-adrenergic receptor agonist will refer to compounds as described in the first aspect of the invention and, as such, may refer to separate compound or a single compound having both activities.

Thus, in relation to such methods and uses, it may be stated that the β₂-adrenergic receptor agonist, or a pharmaceutically acceptable salt thereof, and the β₃-adrenergic receptor agonist, or a pharmaceutically acceptable salt thereof, may be separate compounds or the same compound.

For example, a therapeutically effective amount of a β₂-adrenergic receptor agonist, or a pharmaceutically acceptable salt thereof, and a therapeutically effective amount of a β₃-adrenergic receptor agonist, or a pharmaceutically acceptable salt thereof, as referred to in the third aspect of the invention may refer to administration as separate compounds or as a single compound having both activities.

The skilled person will understand that references to the “treatment of” a particular condition (and similarly “treating”) take their normal meanings in the field of medicine. In particular, the terms may refer to achieving a reduction in the severity of one or more clinical symptom associated with the condition. In particular, the terms may refer to achieving a reduction in the severity of one or more clinical symptom associated with the condition.

As used herein, references to patients will refer to a living subject being treated, including mammalian (e.g. human) patients. In particular embodiments of the relevant aspects of the invention (e.g. the first to third aspects of the invention), the treatment is in a mammal (e.g. a human).

As used herein, the term therapeutically effective amount will refer to an amount of a compound that confers a therapeutic effect on the treated patient. The effect may be objective (i.e. measurable by some test or marker) or subjective (i.e. the subject gives an indication of and/or feels an effect).

As used herein, the term prophylaxis includes references to the prevention of (and, similarly, preventing) the disease or disorder (and vice-versa). As such, references to prevention may also be references to prophylaxis, and vice versa. In particular, the term may refer to achieving a reduction in the likelihood of the patient (or healthy subject) developing the condition (for example, at least a 10% reduction, such as at least a 20%, 30% or 40% reduction, e.g. at least a 50% reduction).

In particular embodiments, references to use in the treatment or prophylaxis will refer in particular to uses in treatment.

For the avoidance of doubt, the methods, pharmaceutical formulations for use, kits-of-parts for use, and uses as described for the first to third aspects of the invention may collectively be referred to as “treatments of the first to third aspects of the invention”.

The skilled person will understand that in the course of the treatments of the first to third aspects of the invention, where they are separate compounds, the β₂-adrenergic receptor agonist, or a pharmaceutically acceptable salt thereof, and the β₃-adrenergic receptor agonist, or a pharmaceutically acceptable salt thereof, may be administered, sequentially, separately and/or simultaneously, over the course of treatment of the relevant condition (i.e. administered in conjunction with each other, as defined for the kits-of-parts of the second aspect of the invention).

For the avoidance of doubt, the term “obesity” as used herein will be understood by those skilled in the art to refer to a condition characterised by abnormal or excessive fat accumulation that may impair health, which conditions will be readily identified by those skilled in the art.

For example, in particular embodiments obesity may be understood to be a condition characterised by abnormal or excessive fat accumulation that may impair health in which the subject (e.g. an adult subject) has a body mass index (BMI) of 30.0 or higher (e.g. 30.0 to 39.9).

In further particular embodiments, the therapeutic method of lowering body fat composition will refer to lowering body fat in the form of adipose tissue.

For avoidance of doubt, the term “metabolic syndrome” as used herein will be understood by those skilled in the art to refer to a condition characterised by a clustering of at least three of the five following medical conditions: abdominal obesity, high blood pressure, high blood sugar, high serum triglycerides and low high-density lipoprotein (HDL) levels, such that the conditions occur together, which conditions will be readily identified by those skilled in the art.

For the avoidance of doubt, the term “dyslipidaemia” as used herein will be understood by those in skilled in the art to refer to a condition characterised by being defined as an abnormal amount of lipids (e.g. cholesterol and/or fat) in the blood (often due to diet and lifestyle), which condition will be readily identified by those skilled in the art.

The skilled person will understand that treatments of the first to third aspects of the invention may further comprise (i.e. be combined with) further (i.e. additional/other) treatment(s) for the same condition.

The skilled person will understand that uses and methods described herein may be achieved by the biological properties provided by the described compounds, such as those described in the examples.

In particular, the uses and methods described herein may be achieved as a result of the ability of the compounds described herein to lower body fat.

Thus, in particular embodiments, the treatment or prophylaxis of obesity, metabolic syndrome and/or dyslipidaemia may be said to be treatment or prophylaxis (e.g. treatment) by lowering body fat (i.e. achieving a reduction in adipose tissue in the patient).

For the avoidance of doubt, the treatment or prophylaxis of obesity may, in certain embodiments, be referred to as the treatment or prophylaxis of obesity by lowering body fat composition and/or reducing body weight.

### Lowering Body Fat

The skilled person will understand that pharmaceutical formulations, kits-of-parts, uses and methods of the invention as defined herein may be useful in lowering body fat composition and/or reducing body weight in a patient (or subject) in need thereof, e.g. a patient who has an above-normal body weight or BMI (e.g. a BMI of 30 or greater), such as in an obese patient, which may be referred to as therapeutically lowering body fat composition and/or reducing body weight.

Alternatively, pharmaceutical formulations of the invention may also be useful in lowering body fat composition and/or reducing body weight in a patient (or subject) who has a normal body weight or BMI. In particular instances, such subjects (e.g. adult subjects) may be non-obese (e.g. having a BMI of less than 30.0), e.g. subjects being overweight (BMI 25.0 to 29.9) or of a healthy weight (BMI 18.5 to 24.9), which may be referred to as non-therapeutically lowering body fat composition and/or reducing body weight. As such, the skilled person will understand that such uses may be performed in patients who are not defined as being obese (e.g. in patients who are defined as being of a healthy weight).

For the avoidance of doubt, references to non-therapeutic uses and methods will refer to uses and methods in patients that are not directed to the treatment of a medical condition but which provide the relevant effects for other purposes, such as for cosmetic purposes.

### Formulations/Methods of Administration

The skilled person will understand that compounds and pharmaceutical formulations as defined herein will normally be administered orally, intravenously, subcutaneously, buccally, rectally, dermally, transdermally, nasally, tracheally, bronchially, sublingually, intranasally, topically, by any other parenteral route or via inhalation, in a pharmaceutically acceptable dosage form. Pharmaceutical formulations as described herein will include compositions in the form of tablets, capsules or elixirs for oral administration, suppositories for rectal administration, sterile solutions or suspensions for parenteral or intramuscular administration, and the like.

In particular embodiments, compounds and pharmaceutical formulations as defined herein are administered orally.

Thus, in particular embodiments, the pharmaceutical formulation(s) is/are provided in a pharmaceutically acceptable dosage form, including tablets or capsules, liquid forms to be taken orally or by injection, suppositories, creams, gels, foams, transdermal patches, plasters, inhalants (e.g. to be applied intranasally). For the avoidance of doubt, in such embodiments, compounds of the invention may be present as a solid (e.g. a solid dispersion), liquid (e.g. in solution) or in other forms, such as in the form of micelles.

In more particular embodiments, the pharmaceutical formulation(s) is/are provided in a pharmaceutically acceptable oral dosage form, including tablets or capsules, which forms may be prepared using techniques known to those skilled in the art.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The skilled person will understand that the pharmaceutical formulation of the invention may be administered (for example, as formulations as described hereinabove) at varying doses, with suitable doses being readily determined by one of skill in the art. Oral, pulmonary and topical dosages (and subcutaneous dosages, although these dosages may be relatively lower) of each active agent may range from between about 0.01 μg/kg of body weight per day (μg/kg/day) to about 20 mg/kg/day of body weight per day (mg/kg/day), preferably about 0.1 μg/kg/day to about 5 mg/kg/day, and more preferably about 1 μg/kg/day to about 2 mg/kg/day (e.g. about 10 μg/kg/day to about 1 mg/kg/day). For example, when administered orally, treatment with such compounds may comprise administration of a formulations typically containing between about 1 μg to about 2000 mg, for example between about 10 μg to about 500 mg, or between 100 μg to about 400 mg (e.g. about 1 mg to about 200 mg or 100 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of from about 10 mg to about 200 mg twice daily) of each active agent.

In any event, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; however, there can, of course, be individual instances where higher or lower dosage ranges are merited, and such are included within the scope of this invention.

As described herein above, the skilled person will understand that the treatments (and methods of prophylaxis) as described here may further comprise (i.e. be combined with) additional (i.e. other) treatment(s) for the same condition. In particular, treatments (and methods of prophylaxis) described herein may be combined with other means for the treatment of excess body weight or a disorder characterized by excess body weight (as defined herein, such as obesity), such as treatment with one or more other therapeutic agent that is useful in the treatment of excess body weight or a disorder characterized by excess body weight (as defined herein, such as obesity).

Such agents will be readily identified by those skilled in the art and include, in particular, such therapeutic agents that are commercially available (e.g. agents that the subject of a marketing authorization in one or more territory, such as a European or US marketing authorization).

### Preparation of Formulations and Kits-of-Parts

Pharmaceutical formulations and kits-of-parts as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a β₂-adrenergic receptor agonist, or a pharmaceutically acceptable salt thereof, and a β₃-adrenergic receptor agonist, or a pharmaceutically acceptable salt thereof, as hereinbefore defined, with one or more pharmaceutically-acceptable excipients (e.g. an adjuvant, diluent and/or carrier).

There is further provided a method of preparing a kit-of-parts as defined hereinbefore, which method comprises bringing component (A) into association with component (B), thus rendering the two components suitable for administration in conjunction with each other. As such, references to bringing into association will mean that the two components are rendered suitable for administration in conjunction with each other.

Thus, in relation to the process for the preparation of a kit-of-parts as hereinbefore defined, by bringing the two components “into association with” each other, it is contemplated that the two components of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

The skilled person will understand that pharmaceutical formulations, kits-of-parts, methods and uses described herein may have the advantage that, in the treatment of the conditions mentioned hereinbefore, they may be more convenient for the physician and/or patient than, be more efficacious than, be less toxic than, have a broader range of activity than, be more potent than, produce fewer side effects than, or may have other useful pharmacological properties over, similar methods (treatments) known in the prior art whether for use in the above-stated indications or otherwise. In particular, such pharmaceutical formulations, kits of parts, methods and uses may have the advantage that they are more efficacious and/or exhibit advantageous properties in vivo.

Without wishing to be bound by theory, it is believed that the β₂-adrenergic receptor and β₃-adrenergic receptor agonists in the pharmaceutical formulations, kits-of-parts, use and methods of the invention (whether separate compounds acting as β₂-agonists and β₃-agonists, or a compound that is both a β₂- and a β₃-agonist) have complementary biological effects which combine to increase the overall level of BAT thermogenesis (i.e. increase above basal levels for a given patient), which in turn results in a reduction in lipid storage in adipose tissue due to lipolysis.

In particular, it is believed that combined activation of the β₂ and β₃ adrenergic receptors, either by the same compound or by a combination of compounds, provides complementary biological effects relating to the distribution of lipids between white adipose tissue (WAT) and brown adipose tissue (BAT), which in turn allows for a potent synergistic effect on the overall level of lipolysis.

Moreover, certain of the compounds acting as β₂-adrenergic receptor agonists, or in some cases as agonists of the β₂- and β₃-adrenergic receptors, as described herein are able to activate the β₂-adrenergic receptor without (or with only a minimal effect in) inducing cAMP production. It is thought that this allows for therapeutic effects as described herein to be obtained with lower levels of side effects than would result from other treatments.

The pharmaceutical formulations of the invention are therefore useful in the treatment or prophylaxis of obesity, lowering body fat composition and/or reducing body weight, the treatment or prophylaxis of metabolic syndrome and the treatment or prophylaxis of dyslipidaemia, and may have advantages over compounds used in such methods as described in the prior art, such as the ability to administer the compound(s) used in such methods of treatment or prophylaxis at lower doses, to achieve greater effects and/or to achieve lower levels of adverse events.

### BRIEF DESCRIPTION OF THE FIGURES

FIG. 1 shows that the glucose uptake promoted by clenbuterol is inhibited in a dose-dependent manner by the selective β₂-adrenergic receptor antagonist ICI-118551.

FIG. 2 shows that the glucose uptake promoted by Compound A is inhibited in a dose-dependent manner by the selective β₂-adrenergic receptor antagonist ICI-118551.

FIG. 3 shows that the glucose uptake promoted by Compound B at a concentration of 1×10⁻⁶ M is inhibited by selective β₂-adrenergic receptor antagonist ICI-118551.

FIG. 4 shows that Compound B promotes cAMP formation.

FIGS. 5 and 6 show a distinct synergistic effect on the reduction of body weight and the lowering of fat mass when the mice are treated with clenbuterol together with CL-316243 compared to when they are treated with clenbuterol or CL-316243 separately.

FIGS. 7 and 8 show a distinct synergistic effect on the reduction of body weight and the lowering of fat mass when the mice are treated with Compound A in combination with the β₃-adrenergic receptor agonist CL-316243 compared to when they are treated with Compound A or CL-316243 separately.

FIGS. 9 and 10 show a distinct reduction of body weight and lowering of fat mass when the mice are treated with Compound B.

### EXAMPLES

The present invention is illustrated by way of the following examples, which are not intended to be limiting on the overall scope of the invention.

For the avoidance of doubt, in the case of a discrepancy between the name of the compound and the structure drawn in this specification, the structure should prevail.

### Biological Example 1: Glucose Uptake in the Presence of a Selective β₂-Adrenergic Receptor Inhibitor

L6-myoblasts were grown in Dulbecco's Modified Eagle's Medium (DMEM) containing 1 g/L glucose supplemented with 10% fetal bovine serum (FBS), 2 mM L-glutamine, 50 U/mL penicillin, 50 g/mL streptomycin and 10 mM HEPES. Cells were plated at 1×10⁵ cells per mL in 24-well plates. After reaching 90% confluence the cells were grown in medium containing 2% FBS for 7 days where upon cells differentiated into myotubes.

The differentiated L6-myotubes were serum-starved overnight in medium containing 0.5% fatty-acid free BSA and stimulated with Compounds A and B at a final concentration of 1×10⁻⁵ M in the presence of the selective β₂-adrenergic receptor antagonist ICI-118551. After 1 h 40 min the cells were washed with warm, glucose free medium twice and another portion of agonist was added to the glucose free medium. After another 20 min of incubation the cells were exposed to 50 nM ³H-2-deoxyglucose for 10 min before washed in ice cold glucose free medium three times and lysed in 400 μl/well 0.2 M NaOH for 1 h at 60° C. The cell lysate was mixed with 4 ml scintillation buffer (Emulsifier Safe, Perkin Elmer) and the radioactivity was detected in a 3-counter (Tri-Carb 4810TR, Perkin Elmer).

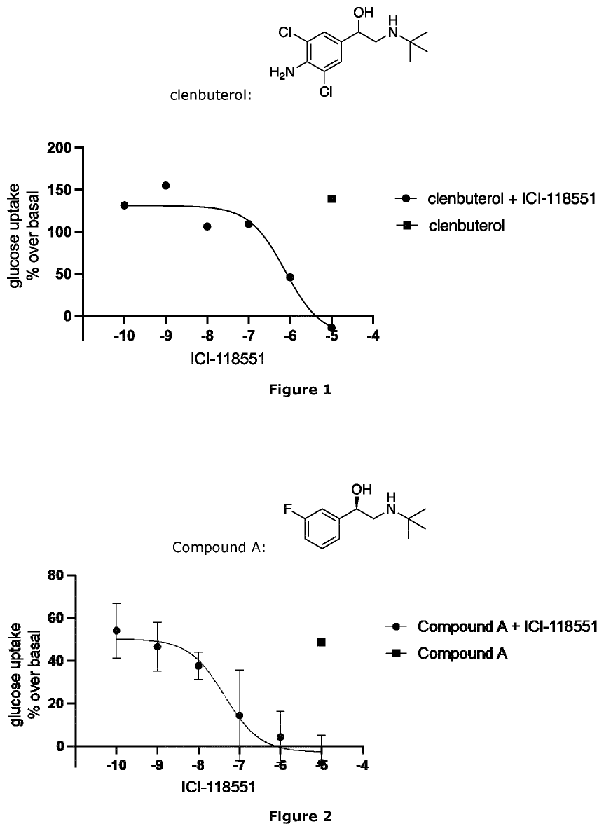

FIG. 1 shows that the glucose uptake promoted by clenbuterol is inhibited in a dose-dependent manner by the selective β₂-adrenergic receptor antagonist ICI-118551, which proves that the glucose uptake promoted by clenbuterol is mediated through the β₂-adrenergic receptor.

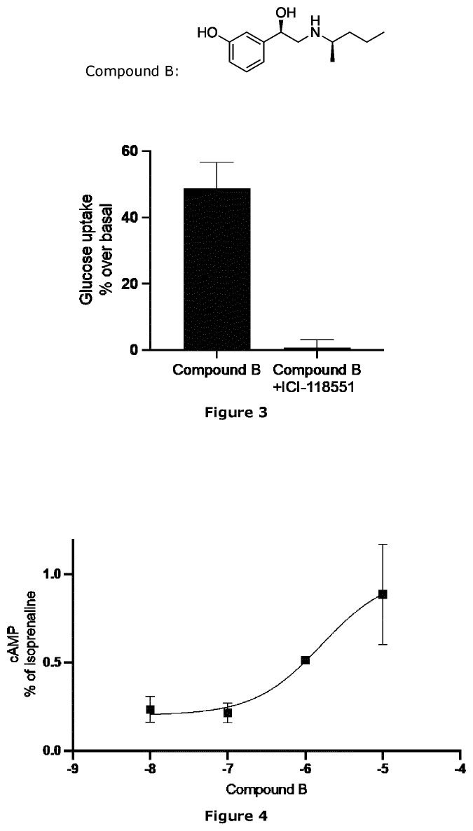

FIG. 2 shows that the glucose uptake promoted by Compound A is inhibited in a dose-dependent manner by the selective β₂-adrenergic receptor antagonist ICI-118551, which proves that the glucose uptake promoted by Compound A is mediated through the β₂-adrenergic receptor.

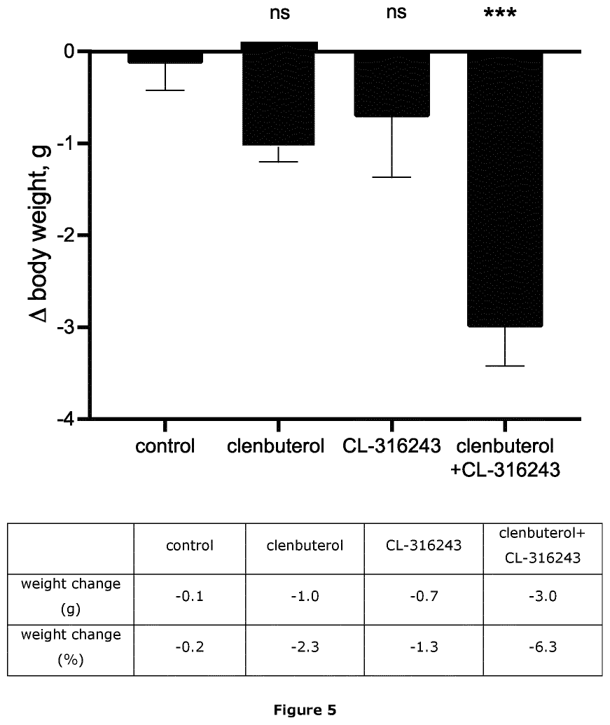

FIG. 3 shows that the glucose uptake promoted by Compound B at a concentration of 1×10⁻⁶ M is inhibited by selective β₂-adrenergic receptor antagonist ICI-118551 at a concentration of 1×10⁻⁶ M, which proves that the glucose uptake promoted by Compound B is mediated through the β₂-adrenergic receptor.

### Biological Example 2: cAMP Levels in CHO-K1 Cells Expressing the Mouse β₃-Adrenergic Receptor

CHO-K1 cells stably expressing the mouse β₃-adrenergic receptors were serum-starved overnight and stimulated with an agonist, final concentration 1×10⁻⁵ M, for 15 min in stimulation buffer (HBSS supplemented with 1% BSA, 5 mM HEPES and 1 mM IBMX, pH 7.4) The medium was then aspirated and to end the reaction, 100 μL of 95% EtOH was added to each well of the 24-well plate and cells were kept at −20° C. overnight. The next day EtOH was allowed to evaporate and 250 μL of lysis buffer (1% BSA, 5 mM HEPES and 0.3% Tween-20, pH 7.4) was added to each well. The plate was kept at −80° C. for 30 min and thawed in room temperature. Intracellular cAMP levels were detected using an alpha screen cAMP kit (6760635D from Perkin Elmer).

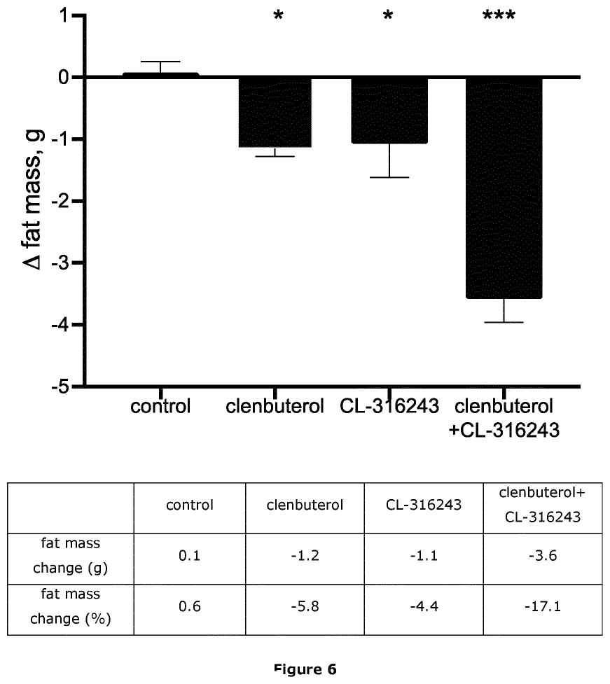

FIG. 4 shows that Compound B in a dose-dependent manner promotes cAMP formation via the β₃-adrenergic receptor in comparison to the positive control isoprenaline.

### Biological Example 3: Fat Mass and Body Weight Reduction with a Combination of Clenbuterol and the β₃-Adrenergic Receptor Agonist CL-316243

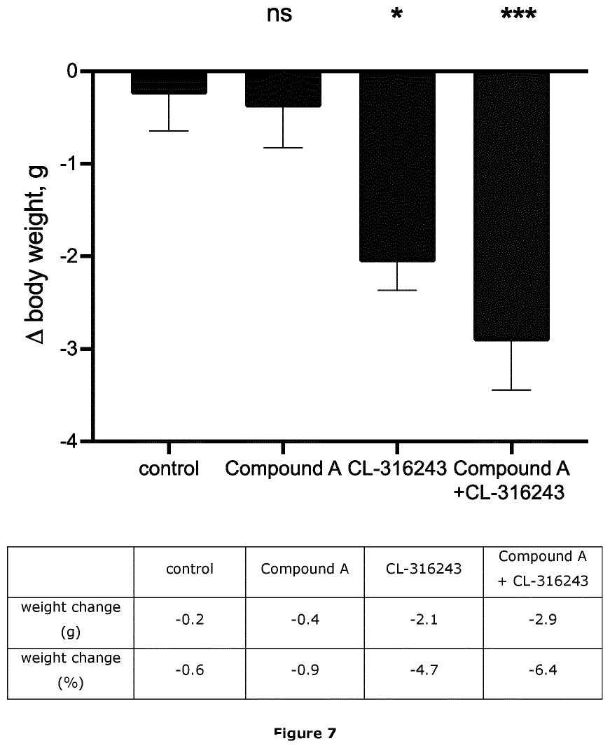

C57Bl/6N mice at an age of 11 weeks were put on 45% high fat diet (ad libitum) for 5 months. The mice were treated by oral gavage with saline, the selective β₂-adrenergic agonist clenbuterol (25 μg/kg), the selective β₃ adrenergic agonist CL-316243 (25 μg/kg) or a combination of clenbuterol (25 μg/kg) and CL-316243 (25 μg/kg) once daily in the morning. After 18 days the mice were weighed and the fat mass was measured by magnetic resonance imaging (EchoMRI-100, Echo Medical Systems). The results are given in FIGS. 5 and 6 and show a distinct synergistic effect on the reduction of body weight and the lowering of fat mass when the mice are treated with clenbuterol together with CL-316243 compared to when they are treated with clenbuterol or CL-316243 separately.

### Biological Example 4: Fat Mass and Body Weight Reduction with a Combination of Compound a and the β₃-Adrenergic Receptor Agonist CL-316243

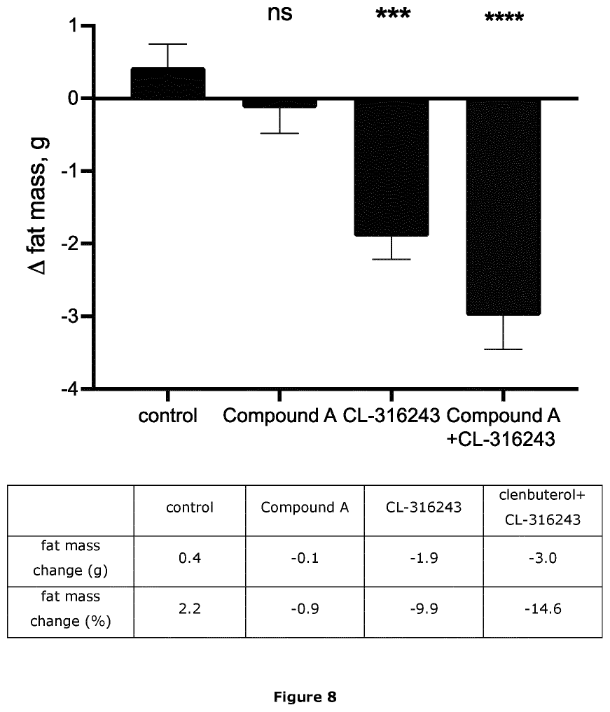

C57Bl/6N mice at an age of 11 weeks were put on 45% high fat diet (ad libitum) for 5 months. The mice were treated by oral gavage with saline, Compound A (300 μg/kg), CL-316243 (25 μg/kg) or a combination Compound A (300 μg/kg) and CL-316243 (25 μg/kg) once daily in the morning. After 18 days the fat mass and the body weight were measured by magnetic resonance imaging (EchoMRI-100, Echo Medical Systems), and by weighing the mice, respectively. The results are given in FIGS. 7 and 8 and show a distinct synergistic effect on the reduction of body weight and the lowering of fat mass when the mice are treated with Compound A in combination with the β₃-adrenergic receptor agonist CL-316243 compared to when they are treated with Compound A or CL-316243 separately.

### Biological Example 5: Fat Mass and Body Weight Reduction with Compound B

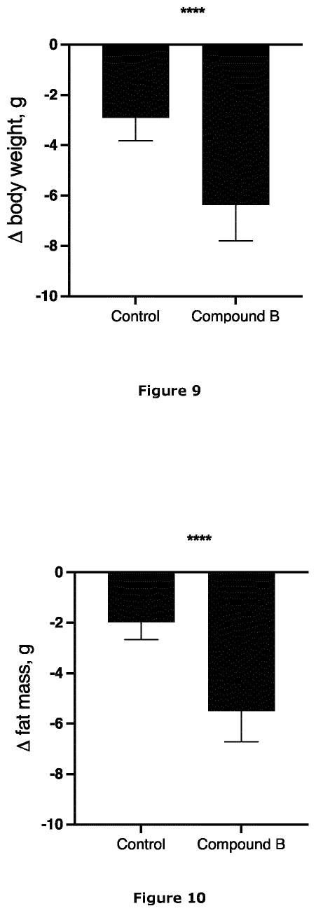

C57Bl/6N mice at an age of 11 weeks were put on 45% high fat diet (ad libitum) for 5 months. The mice were treated by oral gavage with saline or Compound B (5 mg/kg), once daily in the morning. After 21 days the fat mass and the body weight were measured by magnetic resonance imaging (EchoMRI-100, Echo Medical Systems), and by weighing the mice, respectively. The results are given in FIGS. 9 and 10 and show a distinct reduction of body weight and lowering of fat mass when the mice are treated with Compound B.

### Biological Example 6: cAMP Levels in CHO-K1 Cells Expressing the Human β₃-Adrenergic Receptor

CHO-K1 cells stably expressing the human β₃-adrenergic receptors were serum-starved overnight and stimulated with an agonist, final concentration 1×10⁻⁵ M, for 15 min in stimulation buffer (HBSS supplemented with 1% BSA, 5 mM HEPES and 1 mM IBMX, pH 7.4) The medium was then aspirated and to end the reaction, 100 μL of 95% EtOH was added to each well of the 24-well plate and cells were kept at −20° C. overnight. The next day EtOH was allowed to evaporate and 250 μL of lysis buffer (1% BSA, 5 mM HEPES and 0.3% Tween-20, pH 7.4) was added to each well. The plate was kept at −80° C. for 30 min and thawed in room temperature. Intracellular cAMP levels were detected using an alpha screen cAMP kit (6760635D from Perkin Elmer). The activity for each compound is compared to that of isoproterenol. If a compound shows activity of more than 75% of that of isoproterenol at 10 μM the activity is denoted with ++, if it is between 75 and 50% it is denoted with +.

Compounds described in publications referenced and incorporated herein were screened in accordance with the procedure, as indicated in the following table. Synthetic procedures and further biological data (including glucose uptake, which is indicative of activation of the β₂ adrenergic receptor) will be found in the relevant publications.

| | Structure | Name | Activity |
|---|---|---|---|
| 1 | 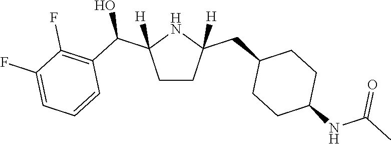 [SMILES: CC(=O)N[C@H]1CC[C@@H](C[C@@H]2CC[C@H]([C@H](O)c3cccc(F)c3F)N2)CC1] | N-((cis)-4-(((2S,5R)-5-((R)- (2,3-difluorophenyl)- (hydroxy)methyl)pyrrolidin-2- yl)methyl)cyclohexyl)- acetamide | ++ |
| 2 | 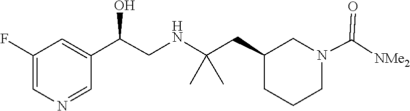 [SMILES: CN(C)C(=O)N1CCC[C@@H](CC(C)(C)NC[C@H](O)c2cncc(F)c2)C1] | (S)-3-(2-(((R)-2-(5-fluoro- pyridin-3-yl)-2-hydroxy- ethyl)amino)-2-methyl- propyl)-N,N-dimethyl- piperidine-1-carboxamide | + |
| 3 | 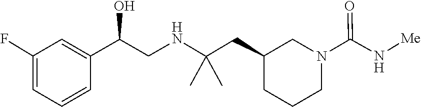 [SMILES: CNC(=O)N1CCC[C@@H](CC(C)(C)NC[C@H](O)c2cccc(F)c2)C1] | (S)-3-(2-(((R)-2-(3- fluorophenyl)-2-hydroxy- ethyl)amino)-2-methyl- propyl)-N-methylpiperidine-1- carboxamide | ++ |
| 4 | 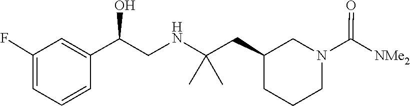 [SMILES: CN(C)C(=O)N1CCC[C@@H](CC(C)(C)NC[C@H](O)c2cccc(F)c2)C1] | (S)-3-(2-(((R)-2-(3-fluoro- phenyl)-2-hydroxyethyl)- amino)-2-methylpropyl)-N,N- dimethylpiperidine-1- carboxamide | ++ |
| 5 | 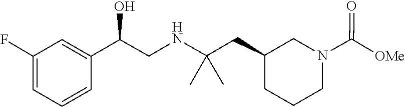 [SMILES: COC(=O)N1CCC[C@@H](CC(C)(C)NC[C@H](O)c2cccc(F)c2)C1] | methyl (S)-3-(2-(((R)-2-(3- fluorophenyl)-2-hydroxy- ethyl)amino)-2-methyl- propyl)piperidine-1- carboxylate | ++ |
| 6 | 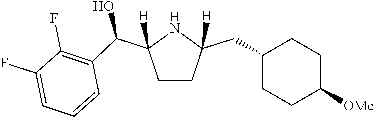 [SMILES: CO[C@H]1CC[C@H](C[C@@H]2CC[C@H]([C@H](O)c3cccc(F)c3F)N2)CC1] | (R)-(2,3-difluorophenyl)- ((2R,5S)-5-(((trans)-4- methoxycyclohexyl)methyl)- pyrrolidin-2-yl)methanol | ++ |
| 7 | 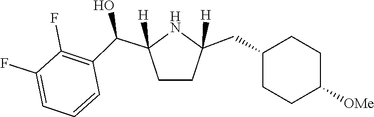 [SMILES: CO[C@H]1CC[C@@H](C[C@@H]2CC[C@H]([C@H](O)c3cccc(F)c3F)N2)CC1] | (R)-(2,3-difluorophenyl)- ((2R,5S)-5-(((cis)-4- methoxycyclohexyl)methyl)- pyrrolidin-2-yl)methanol | + |
| 8 | 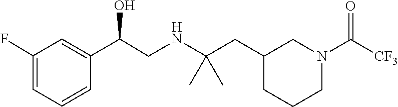 [SMILES: CC(C)(CC1CCCN(C(=O)C(F)(F)F)C1)NC[C@H](O)c1cccc(F)c1] | (R)-1-(3-fluorophenyl)-2-((2- methyl-1-(1-(methyl- sulfonyl)piperidin-4-yl)- propan-2-yl)amino)ethan-1-ol | + |
| 9 | 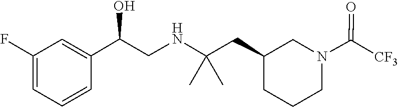 [SMILES: CC(C)(C[C@@H]1CCCN(C(=O)C(F)(F)F)C1)NC[C@H](O)c1cccc(F)c1] | 2,2,2-trifluoro-1-((S)-3-(2- (((R)-2-(3-fluorophenyl)-2- hydroxyethyl)amino)-2- methylpropyl)piperidin-1-yl)- ethan-1-one | + |
| 10 | 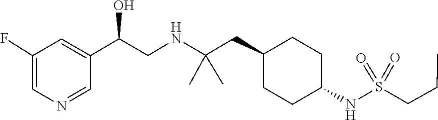 [SMILES: CCCS(=O)(=O)N[C@H]1CC[C@H](CC(C)(C)NC[C@H](O)c2cncc(F)c2)CC1] | N-((trans)-4-(2-(((R)-2-(5- fluoropyridin-3-yl)-2- hydroxyethyl)amino)-2- methylpropyl)cyclohexyl)- propane-1-sulfonamide | ++ |
| 11 | 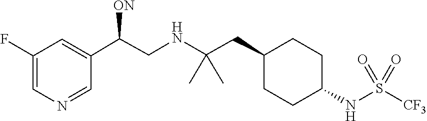 | 1,1,1-trifluoro-N-((1R,4r)-4- (2-(((R)-2-(5-fluoropyridin-3- yl)-2-hydroxyethyl)amino)-2- methylpropyl)cyclohexyl)- methanesulfonamide | ++ |
| 12 | 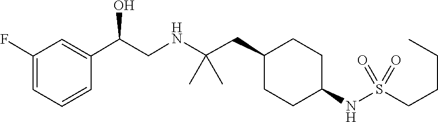 [SMILES: CCCCS(=O)(=O)N[C@H]1CC[C@@H](CC(C)(C)NC[C@H](O)c2cccc(F)c2)CC1] | N-((cis)-4-(2-(((R)-2-(3- fluorophenyl)-2-hydroxy- ethyl)amino)-2-methyl- propyl)cyclohexylbutane-1- sulfonamide | ++ |
| 13 | 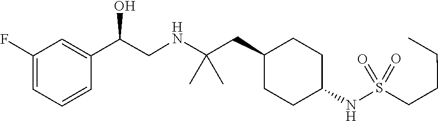 [SMILES: CCCCS(=O)(=O)N[C@H]1CC[C@H](CC(C)(C)NC[C@H](O)c2cccc(F)c2)CC1] | N-((trans)-4-(2-(((R)-2-(3- fluorophenyl)-2-hydroxy- ethyl)amino)-2-methyl- propyl)cyclohexylbutane-1- sulfonamide | ++ |
| 14 | 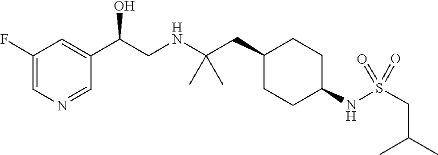 [SMILES: CC(C)CS(=O)(=O)N[C@H]1CC[C@@H](CC(C)(C)NC[C@H](O)c2cncc(F)c2)CC1] | N-((cis)-4-(2-(((R)-2-(5- fluoropyridin-3-yl)-2- hydroxyethyl)amino)-2- methylpropyl)cyclohexyl)-2- methylpropane-1-sulfonamide | + |
| 15 | 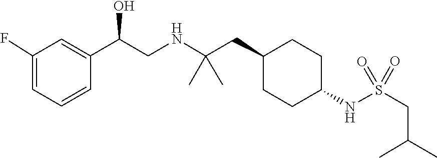 [SMILES: CC(C)CS(=O)(=O)N[C@H]1CC[C@H](CC(C)(C)NC[C@H](O)c2cccc(F)c2)CC1] | N-((trans)-4-(2-(((R)-2-(5- fluoropyridin-3-yl)-2- hydroxyethyl)amino)-2- methylpropyl)cyclohexyl)-2- methylpropane-1-sulfonamide | ++ |
| 16 | 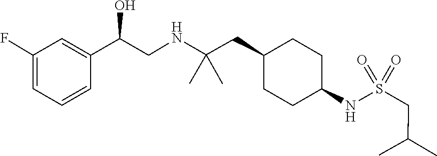 [SMILES: CC(C)CS(=O)(=O)N[C@H]1CC[C@@H](CC(C)(C)NC[C@H](O)c2cccc(F)c2)CC1] | N-((cis)-4-(2-(((R)-2-(3- fluorophenyl)-2-hydroxy- ethyl)amino)-2-methyl- propyl)cyclohexyl)-2-methyl- propane-1-sulfonamide | ++ |
| 17 | 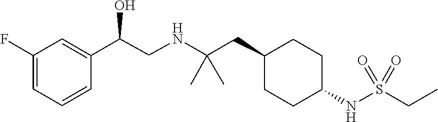 [SMILES: CCS(=O)(=O)N[C@H]1CC[C@H](CC(C)(C)NC[C@H](O)c2cccc(F)c2)CC1] | N-((trans)-4-(2-(((R)-2-(3- fluorophenyl)-2-ethyl)amino)- 2-methylpropyl)cyclohexyl)- ethanesulfonamide | ++ |
| 18 | 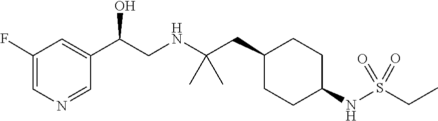 [SMILES: CCS(=O)(=O)N[C@H]1CC[C@@H](CC(C)(C)NC[C@H](O)c2cncc(F)c2)CC1] | N-((cis)-4-(2-(((R)-2-(5- fluoropyridin-3-yl)-2- hydroxyethyl)amino)-2- methylpropyl)cyclo- hexyl)ethanesulfonamide | + + |
| 19 | 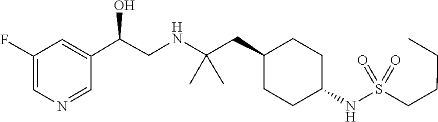 [SMILES: CCCCS(=O)(=O)N[C@H]1CC[C@H](CC(C)(C)NC[C@H](O)c2cncc(F)c2)CC1] | N-((trans)-4-(2-(((R)-2-(5- fluoropyridin-3-yl)-2- hydroxyethyl)amino)-2- methylpropyl)cyclohexyl)- butane-1-sulfonamide | ++ |
| 20 | 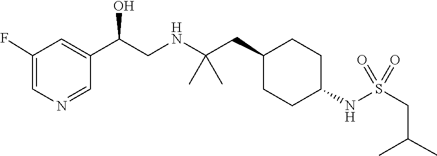 [SMILES: CC(C)CS(=O)(=O)N[C@H]1CC[C@H](CC(C)(C)NC[C@H](O)c2cncc(F)c2)CC1] | N-((trans)-4-(2-(((R)-2-(5- fluoropyridin-3-yl)-2- hydroxyethyl)amino)-2- methylpropyl)cyclohexyl)-2- methylpropane-1-sulfonamide | ++ |
| 21 | 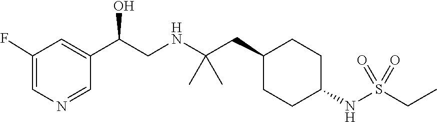 [SMILES: CCS(=O)(=O)N[C@H]1CC[C@H](CC(C)(C)NC[C@H](O)c2cncc(F)c2)CC1] | N-((trans)-4-(2-(((R)-2-(5- fluoropyridin-3-yl)-2- hydroxyethyl)amino)-2- methylpropyl)cyclohexyl)- ethanesulfonamide | ++ |
| 22 | 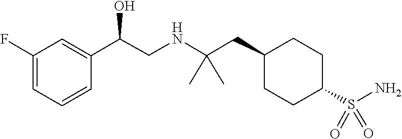 [SMILES: CC(C)(C[C@H]1CC[C@H](S(N)(=O)=O)CC1)NC[C@H](O)c1cccc(F)c1] | (trans)-4-(2-(((R)-2-(3- fluorophenyl)-2-hydroxy- ethyl)amino)-2-methyl- propyl)cyclohexane-1- sulfonamide | ++ |
| 23 | 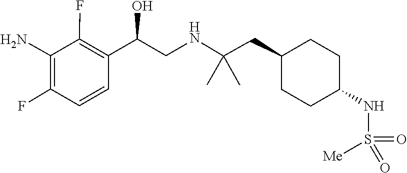 [SMILES: CC(C)(C[C@H]1CC[C@H](NS(C)(=O)=O)CC1)NC[C@H](O)c1ccc(F)c(N)c1F] | N-((trans)-4-(2-(((R)-2-(3- amino-2,4-difluorophenyl)-2- hydroxyethyl)amino)-2- methylpropyl)cyclohexyl)- methanesulfonamide | ++ |
| 24 | 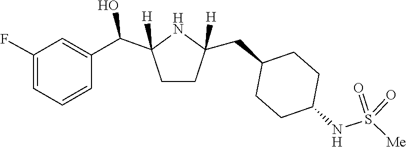 [SMILES: CS(=O)(=O)N[C@H]1CC[C@H](C[C@@H]2CC[C@H]([C@H](O)c3cccc(F)c3)N2)CC1] | N-((trans)-4-(((2S,5R)-5- ((R)-(3-fluorophenyl)- (hydroxy)methyl)pyrrolidin-2- yl)methyl)cyclohexyl)- methanesulfonamide | ++ |
| 25 | 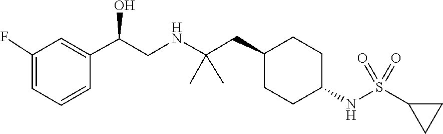 [SMILES: CC(C)(C[C@H]1CC[C@H](NS(=O)(=O)C2CC2)CC1)NC[C@H](O)c1cccc(F)c1] | N-((trans)-4-(2-(((R)-2-(3- fluorophenyl)-2-hydroxy- ethyl)amino)-2-methyl- propyl)cyclohexyl)cyclo- propanesulfonamide | ++ |
| 26 | 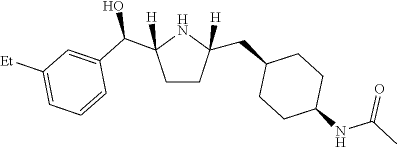 [SMILES: CCc1cccc([C@@H](O)[C@H]2CC[C@@H](C[C@H]3CC[C@@H](NC(C)=O)CC3)N2)c1] | N-((cis)-4-(((2S,5R)-5-((R)- (3-fluorophenyl)(hydroxy)- methyl)pyrrolidin-2-yl)- methyl)cyclohexyl)acetamide | + |
| 27 | 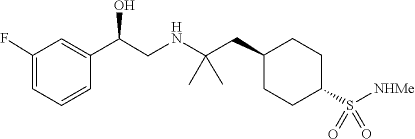 [SMILES: CNS(=O)(=O)[C@H]1CC[C@H](CC(C)(C)NC[C@H](O)c2cccc(F)c2)CC1] | (trans)-4-(2-(((R)-2-(3- fluorophenyl)-2-hydroxy- ethyl)amino)-2-methy- propyl)-N-methyl- cyclohexane-1-sulfonamide | ++ |
| 28 | 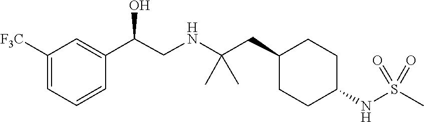 [SMILES: CC(C)(C[C@H]1CC[C@H](NS(C)(=O)=O)CC1)NC[C@H](O)c1cccc(C(F)(F)F)c1] | N-((trans)-4-(2-(((S)-2- hydroxy-2-(6-(trifluoro- methyl)pyridin-2-yl)ethyl)- amino)-2-methylpropyl)- cyclohexyl)methane- sulfonamide | + |
| 29 | 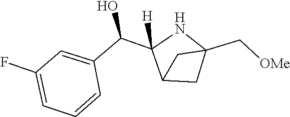 [SMILES: COCC12CC(C1)[C@H]([C@H](O)c1cccc(F)c1)N2] | (R)-(3-fluorophenyl)((R)-1- (methoxymethyl)-2-aza- bicyclo[2.1.1]hexan-3-yl)- methanol | + |
| 30 | 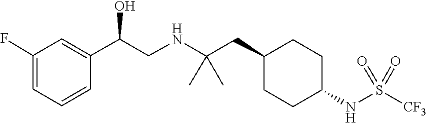 [SMILES: CC(C)(C[C@H]1CC[C@H](NS(=O)(=O)C(F)(F)F)CC1)NC[C@H](O)c1cccc(F)c1] | 1,1,1-trifluoro-N-((trans)-4- (2-(((R)-2-(3-fluorophenyl)- 2-hydroxyethyl)amino)-2- methylpropyl)cyclohexyl)- methanesulfonamide | ++ |
| 31 | 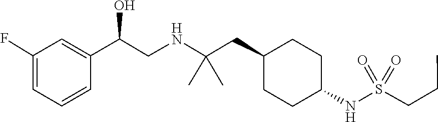 [SMILES: CCCS(=O)(=O)N[C@H]1CC[C@H](CC(C)(C)NC[C@H](O)c2cccc(F)c2)CC1] | N-((trans)-4-(2-(((R)-2-(3- fluorophenyl)-2-hydroxy- ethyl)amino)-2-methyl- propyl)cyclohexyl)propane-1- sulfonamide | ++ |
| 32 | 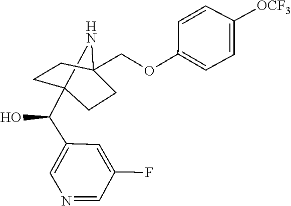 [SMILES: O[C@H](c1cncc(F)c1)C12CCC(COc3ccc(OC(F)(F)F)cc3)(CC1)N2] | (R)-(5-fluoropyridin-3- yl)((1r,4R)-4-((4-(trifluoro- methoxy)phenoxy)methyl)-7- azabicyclo[2.2.1]heptan-1- yl)methanol | ++ |
| 33 | 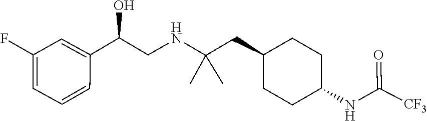 [SMILES: CC(C)(C[C@H]1CC[C@H](NC(=O)C(F)(F)F)CC1)NC[C@H](O)c1cccc(F)c1] | 2,2,2-trifluoro-N-((1R,4r)-4- (2-(((R)-2-(3-fluorophenyl)- 2-hydroxyethyl)amino)-2- methylpropyl)cyclohexyl)- acetamide | ++ |
| 34 | 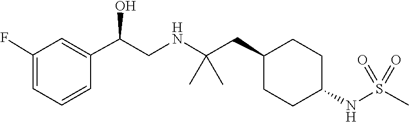 [SMILES: CC(C)(C[C@H]1CC[C@H](NS(C)(=O)=O)CC1)NC[C@H](O)c1cccc(F)c1] | N-((trans-4-(2-(((R)-2-(3- fluorophenyl)-2-hydroxy- ethyl)amino)-2-methyl- propyl)cyclohexyl)- cyclobutanecarboxamide | + |
| 35 | 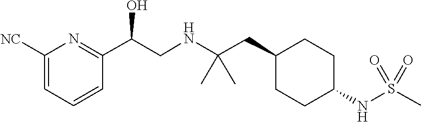 [SMILES: CC(C)(C[C@H]1CC[C@H](NS(C)(=O)=O)CC1)NC[C@H](O)c1cccc(C#N)n1] | N-((trans)-4-(2-(((S)-2-(6- cyanopyridin-2-yl)-2- hydroxyethyl)amino)-2- methylpropyl)cyclohexyl)- methanesulfonamide | ++ |
| 36 | 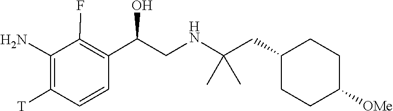 | (R)-1-(3-amino-2,4-difluoro- phenyl)-2-((1-((cis)-4- methoxycyclohexyl)-2- methylpropan-2-yl)amino)- ethan-1-ol | ++ |
| 37 | 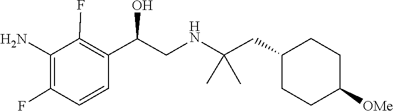 [SMILES: CO[C@H]1CC[C@H](CC(C)(C)NC[C@H](O)c2ccc(F)c(N)c2F)CC1] | (R)-1-(3-amino-2,4-difluoro- phenyl)-2-((1-((trans)-4- methoxycyclohexyl)-2- methylpropan-2-yl)amino)- ethan-1-ol | ++ |
| 38 | 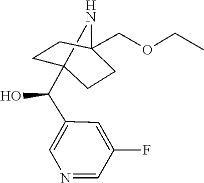 [SMILES: CCOCC12CCC([C@H](O)c3cncc(F)c3)(CC1)N2] | (R)-(4-(ethoxymethyl)-7- azabicyclo[2.2.1]heptan-1- yl)(5-fluoropyridin-3- yl) methanol | + |
| 39 | 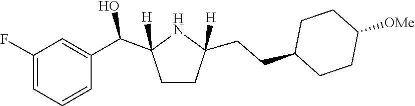 [SMILES: CO[C@H]1CC[C@H](CC[C@@H]2CC[C@H]([C@H](O)c3cccc(F)c3)N2)CC1] | (R)-(3-fluorophenyl)((2R,5R)- 5-(2-((tranbs)-4-methoxy- cyclohexyl)ethyl)pyrrolidin-2- yl)methanol | ++ |
| 40 | 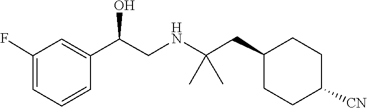 [SMILES: CC(C)(C[C@H]1CC[C@H](C#N)CC1)NC[C@H](O)c1cccc(F)c1] | (trans)-4-(2-(((R)-2-(3- fluorophenyl)-2-hydroxy- ethyl)amino)-2-methyl- propyl)cyclohexane-1- carbonitrile | + |
| 41 | 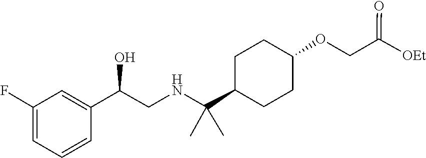 [SMILES: CCOC(=O)CO[C@H]1CC[C@H](C(C)(C)NC[C@H](O)c2cccc(F)c2)CC1] | ethyl 2-(((1R,4r)-4-(2-(((R)- 2-(3-fluorophenyl)-2- hydroxyethyl)amino)propan- 2-yl)cyclohexyl)oxy)acetate | ++ |
| 42 | 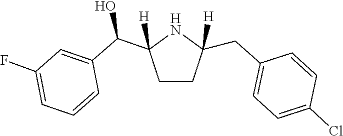 [SMILES: O[C@H](c1cccc(F)c1)[C@H]1CC[C@@H](Cc2ccc(Cl)cc2)N1] | (R)-((2R,5S)-5-(4-chloro- benzyl)pyrrolidin-2-yl)(3- fluorophenyl)methanol | ++ |
| 43 | 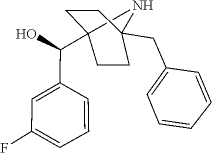 [SMILES: O[C@H](c1cccc(F)c1)C12CCC(Cc3ccccc3)(CC1)N2] | (R)-(4-benzyl-7-azabicyclo- [2.2.1]heptan-1-yl)(3-fluoro- phenyl)methanol | + |
| 44 | 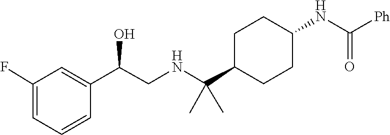 [SMILES: CC(C)(NC[C@H](O)c1cccc(F)c1)[C@H]1CC[C@H](NC(=O)c2ccccc2)CC1] | N-((trans)-4-(2-(((R)-2-(3- fluorophenyl)-2-hydroxy- ethyl)amino)propan-2-yl)- cyclohexyl)benzamide | ++ |
| 45 | 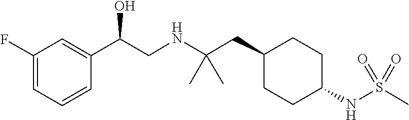 [SMILES: CC(C)(C[C@H]1CC[C@H](NS(C)(=O)=O)CC1)NC[C@H](O)c1cccc(F)c1] | N-((trans)-4-(2-(((R)-2-(3- fluorophenyl)-2-hydroxy- ethyl)amino)-2-methyl- propyl)cyclohexyl)methane- sulfonamide | ++ |
| 46 | 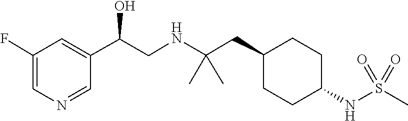 [SMILES: CC(C)(C[C@H]1CC[C@H](NS(C)(=O)=O)CC1)NC[C@H](O)c1cncc(F)c1] | N-((trans)-4-(2-(((R)-2-(5- fluoropyridin-3-yl)-2- hydroxyethyl)amino)-2- methylpropyl)cyclohexyl) methanesulfonamide | ++ |
| 47 | 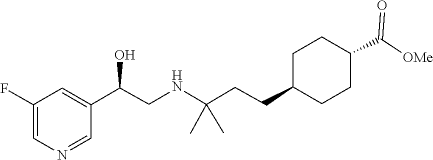 [SMILES: COC(=O)[C@H]1CC[C@H](CCC(C)(C)NC[C@H](O)c2cncc(F)c2)CC1] | methyl (trans)-4-(3-(((R)-2- (5-fluoropyridin-3-yl)-2- hydroxyethyl)amino)-3- methylbutyl)cyclohexane-1- carboxylate | + |
| 48 | 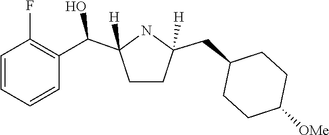 | (R)-(2-fluorophenyl)((2R,5R)- 5-(((trans)-4-methoxycyclo- hexyl)methyl)pyrrolidin-2-yl)- methanol | + |
| 49 | 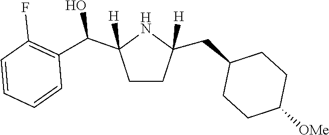 [SMILES: CO[C@H]1CC[C@H](C[C@@H]2CC[C@H]([C@H](O)c3ccccc3F)N2)CC1] | (R)-(2-fluorophenyl)((2R,5S)- 5-(((trans)-4-methoxycyclo- hexyl)methyl)pyrrolidin-2-yl)- methanol | + |
| 50 | 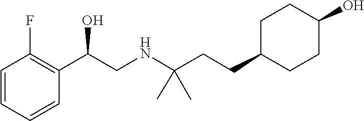 [SMILES: CC(C)(CC[C@H]1CC[C@@H](O)CC1)NC[C@H](O)c1ccccc1F] | (cis)-4-(3-(((R)-2-(2-fluoro- phenyl)-2-hydroxyethyl)- amino)-3-methylbutyl)- cyclohexan-1-ol | + |
| 51 | 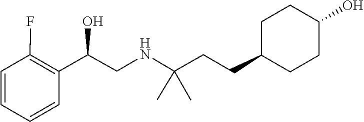 [SMILES: CC(C)(CC[C@H]1CC[C@H](O)CC1)NC[C@H](O)c1ccccc1F] | (trans)-4-(3-(((R)-2-(2- fluorophenyl)-2-hydroxy- ethyl)amino)-3-methylbutyl)- cyclohexan-1-ol | ++ |
| 52 | 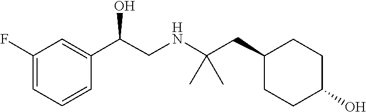 [SMILES: CC(C)(C[C@H]1CC[C@H](O)CC1)NC[C@H](O)c1cccc(F)c1] | (trans)-4-(2-(((R)-2-(3- fluorophenyl)-2-hydroxy- ethyl)amino)-2-methyl- propyl)cyclohexan-1-ol | ++ |
| 53 | 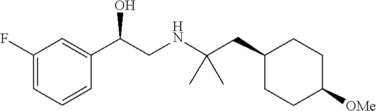 [SMILES: CO[C@H]1CC[C@@H](CC(C)(C)NC[C@H](O)c2cccc(F)c2)CC1] | (R)-1-(3-fluorophenyl)-2-((1- ((cis)-4-methoxycyclohexyl)- 2-methylpropan-2-yl)- amino)ethan-1-ol | + |
| 54 | 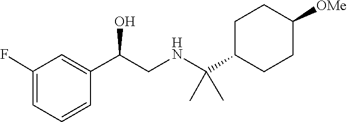 [SMILES: CO[C@H]1CC[C@H](C(C)(C)NC[C@H](O)c2cccc(F)c2)CC1] | (R)-1-(3-fluorophenyl)-2-((2- ((trans)-4-methoxycyclo- hexyl)propan-2-yl)amino)- ethan-1-ol | + |
| 55 | 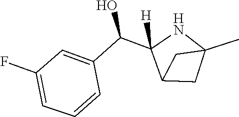 [SMILES: CC12CC(C1)[C@H]([C@H](O)c1cccc(F)c1)N2] | (R)-(3-fluorophenyl)((R)-1- methyl-2-azabicyclo[2.1.1]- hexan-3-yl)methanol | + |
| 56 | 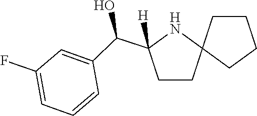 [SMILES: O[C@H](c1cccc(F)c1)[C@H]1CCC2(CCCC2)N1] | (R)-(3-fluorophenyl)((R)-1- azaspiro[4.4]nonan-2-yl)- methanol | ++ |
| 57 | 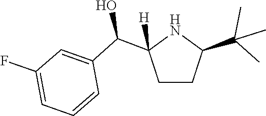 [SMILES: CC(C)(C)[C@H]1CC[C@H]([C@H](O)c2cccc(F)c2)N1] | (R)-((2R,5R)-5-(tert-butyl)- pyrrolidin-2-yl)(3-fluoro- phenyl)methanol | + |
| 58 | 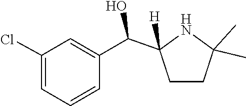 [SMILES: CC1(C)CC[C@H]([C@H](O)c2cccc(Cl)c2)N1] | (R)-(3-chlorophenyl)((R)-5,5- dimethylpyrrolidin-2-yl)- methanol | + |
| 59 | 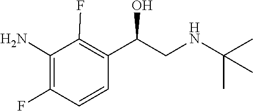 [SMILES: CC(C)(C)NC[C@H](O)c1ccc(F)c(N)c1F] | (R)-1-(3-amino-2,4-difluoro- phenyl)-2-(tert-butylamino)- ethan-1-ol | ++ |
| 60 | 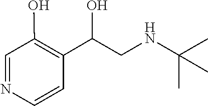 | 4-(2-(tert-butylamino)-1- hydroxyethyl)pyridin-3-ol | ++ |
| 61 | 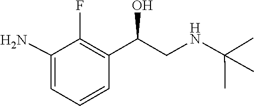 [SMILES: CC(C)(C)NC[C@H](O)c1cccc(N)c1F] | (R)-1-(3-amino-2-fluoro- phenyl)-2-(tert-butylamino)- ethan-1-o | ++ |
| 62 | 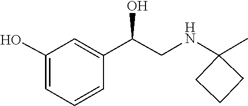 [SMILES: CC1(NC[C@H](O)c2cccc(O)c2)CCC1] | (R)-3-(1-hydroxy-2-((1- methylcyclobutyl)amino)- ethyl)phenol | ++ |
| 63 | 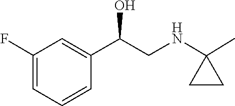 | (R)-1-(3-fluorophenyl)-2-((1- methylcyclopropyl)amino)- ethan-1-ol | + |
| 64 | 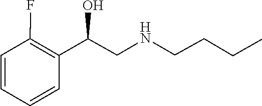 | (R)-2-(butylamino)-1-(2- fluorophenyl)ethan-1-ol | ++ |
| 65 | 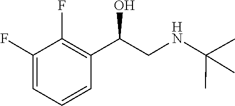 [SMILES: CC(C)(C)NC[C@H](O)c1cccc(F)c1F] | (R)-2-(tert-butylamino)-1- (2,3-difluorophenyl)ethan-1- ol | ++ |
| 66 | 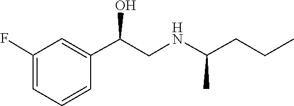 [SMILES: CCC[C@@H](C)NC[C@H](O)c1cccc(F)c1] | (R)-1-(3-fluorophenyl)-2- (((R)-pentan-2-yl)amino)- ethan-1-ol | ++ |
| 67 | 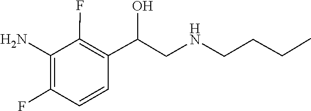 [SMILES: CCCCNCC(O)c1ccc(F)c(N)c1F] | 1-(3-amino-2,4-difluoro- phenyl)-2-(butylamino)ethan- 1-ol | + |
| 68 | 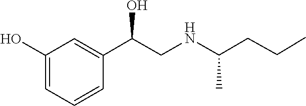 [SMILES: CCC[C@H](C)NC[C@H](O)c1cccc(O)c1] | 3-((R)-1-hydroxy-2-(((S)- pentan-2-yl)amino)ethyl)- phenol | ++ |
| 69 | 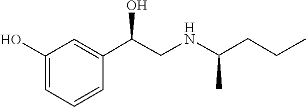 [SMILES: CCC[C@@H](C)NC[C@H](O)c1cccc(O)c1] | 3-((R)-1-hydroxy-2-(((R)- pentan-2-yl)amino)ethyl)- phenol | ++ |
| 70 | 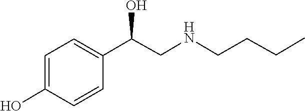 | (R)-4-(2-(butylamino)-1- hydroxyethyl)phenol | + |

1. A pharmaceutical formulation comprising one or more compounds that are:
(a) a β₂-adrenergic receptor agonist, or a pharmaceutically acceptable salt thereof; and
(b) a β₃-adrenergic receptor agonist, or a pharmaceutically acceptable salt thereof,
and optionally one or more pharmaceutically acceptable excipient, for use in:
(i) the treatment or prophylaxis of obesity;
(ii) a therapeutic method of lowering body fat composition and/or reducing body weight;
(iii) the treatment or prophylaxis of metabolic syndrome; or
(iv) the treatment or prophylaxis of dyslipidaemia.

2. A method for:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia,
comprising administering to a patient in need thereof a therapeutically effective amount of a pharmaceutical formulation comprising one or more compounds that are:
(a) a β₂-adrenergic receptor agonist, or a pharmaceutically acceptable salt thereof; and
(b) a β₃-adrenergic receptor agonist, or a pharmaceutically acceptable salt thereof, and optionally one or more pharmaceutically acceptable excipient.

3. A kit-of-parts comprising components:
(A) a pharmaceutical formulation comprising a β₂-adrenergic receptor agonist, or a pharmaceutically acceptable salt thereof, optionally in admixture with one or more pharmaceutically acceptable excipient, and
(B) a pharmaceutical formulation comprising a β₃-adrenergic receptor agonist, or a pharmaceutically acceptable salt thereof, optionally in admixture with one or more pharmaceutically acceptable excipient; or
(C) a pharmaceutical formulation comprising a compound that is a β₂-adrenergic receptor agonist, or a pharmaceutically acceptable salt thereof, and a β₃-adrenergic receptor agonist, or a pharmaceutically acceptable salt thereof, optionally in admixture with one or more pharmaceutically acceptable excipient,
which components (A) and (B) are each provided in a form that is suitable for administration in conjunction with the other, for use in:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia.

4. A kit-of-parts comprising:
(I) one of components (A) or (B) as described in claim 3, and
(II) instructions to use that component in conjunction with the other of the two components, for use in:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia.

5. A β₂-adrenergic receptor agonist, or a pharmaceutically acceptable salt thereof, for use in:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia,
wherein the use also comprises administration of a β₃-adrenergic receptor agonist, or a pharmaceutically acceptable salt thereof.

6. A β₃-adrenergic receptor agonist, or a pharmaceutically acceptable salt thereof, for use in:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia,
wherein the use also comprises administration of a β₂-adrenergic receptor agonist, or a pharmaceutically acceptable salt thereof.

7. A method for:
(i) the treatment or prophylaxis of obesity;
(ii) lowering body fat composition and/or reducing body weight;
(iii) the treatment or prophylaxis of metabolic syndrome; or
(iv) the treatment or prophylaxis of dyslipidaemia,
comprising administration of a therapeutically effective amount of a β₂-adrenergic receptor agonist, or a pharmaceutically acceptable salt thereof, and administration of a therapeutically effective amount of a β₃-adrenergic receptor agonist, or a pharmaceutically acceptable salt thereof, to a patient in need thereof.

8. The formulation for use, kit-of-parts for use, use or method of any one of claims 1 to 7, wherein the β₂-adrenergic receptor agonist, or a pharmaceutically acceptable salt thereof, and the β₃-adrenergic receptor agonist, or a pharmaceutically acceptable salt thereof, are separate compounds.

9. The formulation for use, kit-of-parts for use, use or method of claim 8, wherein the β₂-adrenergic receptor agonist is clenbuterol.

10. The formulation for use, kit-of-parts for use, use or method of claim 8 or claim 9, wherein the β₃-adrenergic receptor agonist is CL-316243.

11. The formulation for use, kit-of-parts for use, use or method of any one of claims 1 to 7, wherein the β₂-adrenergic receptor agonist, or a pharmaceutically acceptable salt thereof, and the β₃-adrenergic receptor agonist, or a pharmaceutically acceptable salt thereof, are the same compound.
